(19) United States
(12) Patent Application Publication
(10) Pub. No.: US 2026/0188481 A1
(43) Pub. Date: Jul. 2, 2026

(54) PHYSIOLOGICAL INFORMATION APPLICATION PLATFORM

(71) Applicant: BROADSIMS INC., Zhubie City (TW)

(72) Inventors: JUI-YUAN YU, Hsinchu City (TW); PENG-HSIANG WANG, Zhubei City (TW)

(21) Appl. No.: 19/431,126

(22) Filed: Dec. 23, 2025

(30) Foreign Application Priority Data
Dec. 31, 2024 (TW) 113151712

(51) Int. Cl. G16H 40/63; A61B 5/00

(52) U.S. Cl. CPC G16H 40/63; A61B 5/0022; A61B 5/742; A61B 5/746

(57) ABSTRACT

A physiological information application platform includes a physiological information application device. The physiological information application device provides data exchange and utilization between a client application and a server application. Before exchanging data between the client application and the server application, it is required to confirm that the applications are authenticated as trusted applications, and an authentication is performed by obtaining an authorized encryption file through an authorization mechanism. Therefore, the physiological information application device provides a data topology application and an alarm management mechanism within an alarm system. Furthermore, the physiological information application device can operate without transferring the data to an external system, thereby reducing a risk of data loss and theft and improving data security.

### CROSS-REFERENCE TO RELATED APPLICATION(S)

This application claims the priority benefit of TW application No. 113151712 filed on Dec. 31, 2024, the entirety of which is hereby incorporated by reference herein and made a part of the specification.

### BACKGROUND OF THE INVENTION

### 1. Field of the Invention

The present invention relates to an application platform, more particularly a physiological information application platform.

### 2. Description of the Related Art

With the development of computer technology, applications of medical measurement data and manually evaluated data have rapidly advanced. As hospitals pursue paperless operation and continuous optimization of workflow, a requirement to collect and upload data to hospital information systems has become very common. However, since each hospital has its own information system and communication protocol, difficulties and obstacles are encountered when performing data interfacing and integration. Moreover, with vigorous development of artificial intelligence (AI) and improvement of AI inference capabilities, AI-based auxiliary diagnosis and early warning software have also emerged. Similarly, the software also encounters the difficulties and the obstacles in data interfacing and integration.

In general, when a medical device collects signal data, numerical data, audio data, or image data, to perform subsequent applications, the collected data must be transferred to an external information device, and the external information device executes a corresponding software to process the collected data. For example, the collected data may be transmitted to a hospital information system. The hospital information system further transfers the collected data to an external information device having corresponding auxiliary diagnostic analysis software or early warning software to complete nursing assessment forms, such as forms for coma index, limb muscle strength, pupil size, and other nursing evaluations. Since the hospital information system still needs to transfer the collected data to the external information device installed with corresponding software, it is difficult to integrate the collected data. Furthermore, complexity of medical and nursing operations is increased, equipment management is complicated, and data insecurity is also an issue.

Accordingly, there is a need for a physiological information application device to improve the above-mentioned drawbacks, including difficulties in data integration, complexity of medical and nursing operations, complexity of equipment management, and insecurity of data.

### SUMMARY OF THE INVENTION

In view of the above-mentioned issues, the present invention provides a physiological information application platform to improve drawbacks such as difficulties in data integration, complexity of medical and nursing operations, complexity of equipment management, and insecurity of data.

The physiological information application platform includes a physiological information application device. The physiological information application device includes a sensing signal receiver, a memory, and a processor. The sensing signal receiver is communicatively connected to a plurality of sensors for receiving a plurality of physiological data. The memory stores at least one server application program and at least one client application program.

The processor is connected to the sensing signal receiver and the memory for accessing the server application program and the client application program. The processor executes the server application program to collect the physiological data. The processor further executes the client application program to access the physiological data collected by executing the server application program, and to generate application program information according to the physiological data.

The processor of the physiological information application device of the physiological information application platform collects the physiological data by executing the server application program, and accesses the physiological data by executing the client application program. Therefore, the client application program can use the physiological data to generate the application program information. Since the physiological data are not transferred to an external information device and are exchanged only within the physiological information application device, data security can be enhanced. Furthermore, because the physiological data are processed within the one physiological information application device, equipment management can be simplified.

### BRIEF DESCRIPTION OF THE DRAWINGS

FIG. 1 is a block diagram of a physiological information application platform of the present invention.

FIG. 2 is a schematic diagram of a star topology architecture of application programs of a physiological information application device of the physiological information application platform of the present invention.

FIG. 3 is a schematic diagram of a tree topology architecture of the application programs of the physiological information application device of the physiological information application platform of the present invention.

### DETAILED DESCRIPTION OF THE INVENTION

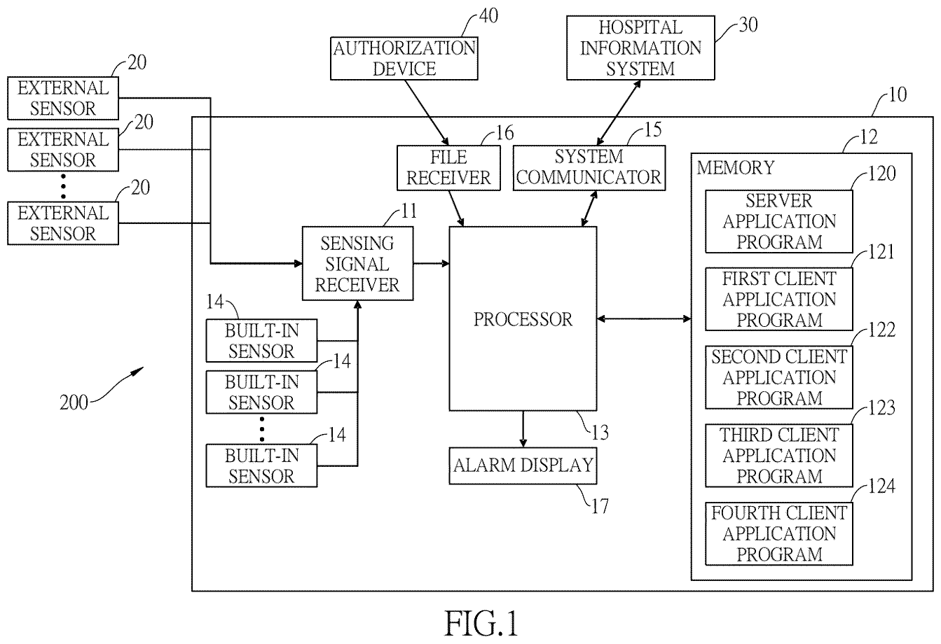

With reference to FIG. 1, a physiological information application platform includes a physiological information application device 10. The physiological information application device 10 includes a sensing signal receiver 11, a memory 12, and a processor 13.

The sensing signal receiver 11 is communicatively connected to a plurality of sensors 200 for receiving a plurality of physiological data generated by the sensors 200. The memory 12 stores a server application program 120 and client application programs 121-124. The processor 13 is connected to the sensing signal receiver 11 and the memory 12 for accessing the server application program 120 and the client application programs 121-124.

The processor 13 executes the server application program 120 to collect the physiological data. The processor 13 further executes the client application programs 121-124 to access the physiological data collected by executing the server application program 120. The processor 13 generates the application program information according to the physiological data. Then, the application program information can be provided to a client for utilizing the physiological data.

Since the physiological data are exchanged only within the physiological information application device 10, the physiological data are not transferred to an external information device. In addition, because the physiological data are processed within the physiological information application device 10, equipment management can be simplified. Further, as the physiological data are exchanged only within the physiological information application device 10 without being transferred to an external information device, data security can also be enhanced.

Furthermore, the sensors 200 include a plurality of built-in sensors 14, and the built-in sensors 14 are mounted in the physiological information application device 10. Namely, the physiological information application device 10 includes the built-in sensors 14. The built-in sensors 14 are connected to the sensing signal receiver 11, and the sensing signal receiver 11 receives the physiological data generated by the built-in sensors 14.

The sensors 200 further include a plurality of external sensors 20. The sensing signal receiver 11 is communicatively connected to the external sensors 20, and receives the physiological data generated by the external sensors 20.

In addition, the physiological information application device 10 further includes a system communicator 15, and the processor 13 is further connected to the system communicator 15. The system communicator 15 is communicatively connected to a hospital information system 30. The client application programs 121-124 at least include a first client application program 121. The processor 13 executes the first client application program 121 to access the physiological data collected by executing the server application program 120, and the processor 13 generates first application program information based on the physiological data. The processor 13 further transmits the first application program information to the hospital information system 30 through the system communicator 15.

Furthermore, the physiological information application device 10 further includes a file receiver 16. The file receiver 16 is communicatively connected to an authorization device 40 for receiving an authorization encrypted file. The memory 12 further stores a plurality of friendly application programs. The processor 13 is connected to the file receiver 16 to receive the authorization encrypted file, and the processor 13 further decrypts the authorization encrypted file to generate authorization verification information. Furthermore, the processor 13 accesses one of the friendly application programs, and generates friendly application program information according to the friendly application program. The processor 13 further determines whether the authorization verification information is same as the friendly application program information. When the authorization verification information is same as the friendly application program information, the processor 13 executes the friendly application program to access the physiological data collected by executing the server application program, and generates the application program information based on the physiological data. For example, the processor 13 may execute the friendly application program to exchange data with the server application program 120 or the client application programs 121-124. Alternatively, the processor 13 may execute the friendly application program to access the physiological data collected by executing the server application program 120. The processor 13 further generates the friendly application program information, according to the physiological data, and transmits the friendly application program information to the hospital information system 30 through the system communicator 15.

In an embodiment, the authorization encrypted file includes an authenticated application identification number and an application version. For example, the authorization verification information generated by the processor 13 through decryption of the authorization encrypted file is the authenticated application identification number and the authenticated application version. In addition, the friendly application program information generated by the processor 13 according to the friendly application program is an application identification number and an application version of the friendly application program. The processor 13 compares the authenticated application identification number and the authenticated application version with the application identification number and the application version of the friendly application program to determine whether they are the same. Accordingly, the friendly application program can be installed under an authorized condition, and the processor 13 can execute the authorized friendly application program. Namely, application programs are not allowed to be installed arbitrarily, thereby preventing intrusions of malicious programs. Therefore, the physiological information application device 10 can provide a data exchange mechanism for exchanging data within an alarm system in a controlled environment.

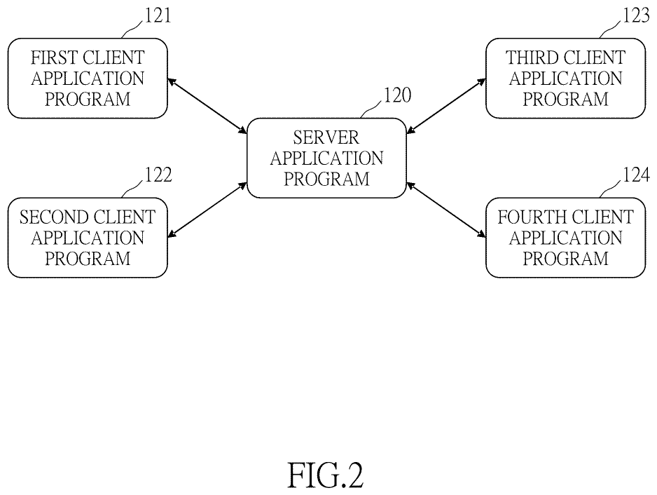

With reference to FIG. 2, in one embodiment, the memory 12 stores the plurality of client application programs 121-124, and the client application programs 121-124 at least include the first client application program 121 and a second client application program 122. The processor 13 executes the first client application program 121 to access the physiological data collected by executing the server application program 120, and the processor 13 further generates first application program information by calculating the physiological data. Moreover, the processor 13 executes the second client application program 122 to access the physiological data collected by executing the server application program 120, and the processor 13 generates second application program information by calculating the physiological data.

Furthermore, the client application programs 121-124 may further include a third client application program 123 and a fourth client application program 124. Similarly, the processor 13 executes the third client application program 123 to access the physiological data collected by executing the server application program 120, and the processor 13 generates third application program information by calculating the physiological data. The processor 13 further executes the fourth client application program 124 to access the physiological data collected by executing the server application program 120, and the processor 13 generates fourth application program information by calculating the physiological data.

In the embodiment, the server application program 120 and the first to fourth client application programs 121-124 form a star topology architecture. The first to fourth client application programs 121-124 each operate independently and exchange data only with the server application program 120. Namely, the first to fourth client application programs 121-124 can simultaneously obtain data from the server application program 120, and perform respective applications. For example, the first client application program 121 is an arrhythmia auxiliary diagnosis program to determine arrhythmia according to the physiological data collected by the server application program 120. The second client application program 122 is an interface program to generate a simplified user interface and to perform large-font display according to the physiological data collected by the server application program 120. The third client application program 123 is a pain index calculation program to calculate a pain index according to the physiological data collected by the server application program 120. The fourth client application program 124 is a stroke index calculation program to calculate a stroke index according to the physiological data collected by the server application program 120.

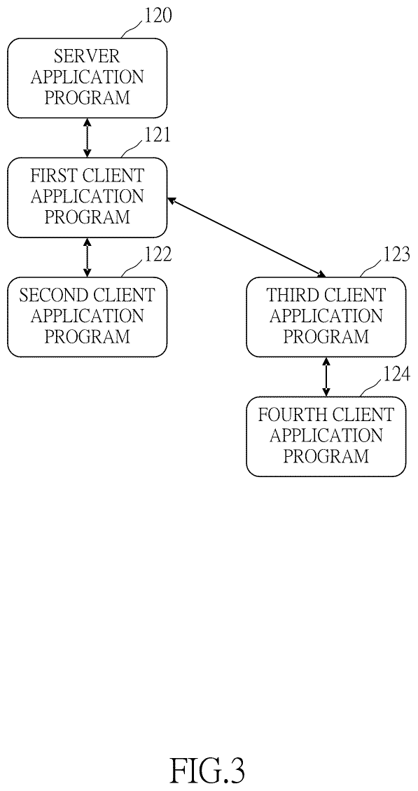

With reference to FIG. 3, in another embodiment, the memory 12 also stores the plurality of client application programs 121-124, and the client application programs 121-124 at least include the first client application program 121 and a second client application program 122. In the embodiment, the processor 13 executes the first client application program 121 to access the physiological data collected by executing the server application program 120, and the processor 13 generates first application program information, according to the physiological data. The processor 13 executes the second client application program 122 to access the first application program information generated by executing the first client application program 121, and the processor 13 generates second application program information by calculating the first application program information.

Furthermore, the client application programs may further include a third client application program 123 and a fourth client application program 124. Similarly, the processor 13 executes the third client application program 123 to access the first application program information generated by executing the first client application program 121, and the processor 13 generates third application program information according to the first application program information. The processor 13 executes the fourth client application program 124 to access the third application program information generated by executing the third client application program 123, and the processor 13 generates the third application program information by calculating the third application program information.

In the embodiment, the server application program 120 and the first to fourth client application programs 121-124 form a tree topology architecture. Therefore, the first to fourth application program information generated by the first to fourth client application programs 121-124 can be superimposed and then applied. For example, the first client application program 121 is a nursing assessment form filling and collection program, and the first client application program 121 fills and collects the nursing assessment forms according to the physiological data collected by the server application program 120. The second client application program 122 is the interface program configured to generate the simplified user interface, and display a large-font image according to the nursing assessment forms generated by the first client application program 121. The third client application program 123 is an alarm program, and the third client application program 123 superimposes the physiological data collected by the server application program 120 with the nursing assessment forms generated by the first client application program 121. Then, the third client application program 123 executes an early warning algorithm to generate alarm information. The fourth client application program 124 is an upload program, and the fourth client application program 124 uploads the physiological data collected by the server application program 120, the nursing assessment forms generated by the first client application program 121, and the alarm information generated by the third client application program 123 to the hospital information system 30.

By means of the tree topology architecture, the information generated by the first to fourth client application programs 121-124 can be flexibly replaced and interconnected to meet various clinical requirements. Therefore, problems of difficulty in data integration can be mitigated, facilitating data re-utilization.

Furthermore, the physiological information application device 10 further includes an alarm display 17, and the alarm display 17 is connected to the processor 13. The memory 12 further stores alarm system parameter information. The application program information generated by the processor 13 is alarm information.

The processor 13 calculates an alarm system utilization rate according to a number of occurrences of the alarm information generated within each unit time and the alarm system parameter information. When the alarm system utilization rate is greater than a utilization threshold, the processor 13 generates the alarm information according to the alarm system utilization rate, and the processor 13 transmits the alarm information to the alarm display 17.

The alarm information can be classified into high-level, medium-level, and low-level alarm information according to their respective levels. The high-level alarm information means information that must be promptly brought to attention of a user. However, since limitations of visual and auditory presentation in space and time, it is difficult to provide a better user experience. Further, in view of the fact that occurrence of alarms is random and memoryless, an M/M/c queuing model is adopted to calculate an alarm queue length and an alarm waiting time, and to allow adjustments of the alarm information. In the M/M/c queuing model, the first “M” indicates Markovian arrival, which means an arrival of the alarm is random and memoryless. Namely, arrival time intervals of the alarms follow an exponential distribution, and an arrival process is a Poisson process. The second “M” indicates Markovian alarm display time, which means a display time of the alarm is also random and memoryless. Further, the alarm display time may be a time for visual or auditory presentation of the alarm. The “c” indicates a number of alarm service stations, that is, a number of the alarms that can be visually or audibly presented simultaneously in the alarm system.

The adjustments of the alarm information include the following:

Increasing the number of the alarm service stations.

Reducing the display time of each alarm to increase a service rate, and the display time of each alarm must be greater than a minimum acceptable time.

Displaying only the high-level alarms, and filtering out medium-level and low-level alarms to reduce the number of the arrivals of the alarms and thereby reduce an arrival rate of the alarm information.

In the embodiment, the alarm system utilization rate is calculated according to the following equation:

$$ρ = \frac{λ}{c \times μ}$$

ρ indicates the alarm system utilization rate, A indicates the number of the occurrences of the alarm information generated per minute, c indicates a display number of simultaneously displaying the alarm information by the alarm display 17, and u indicates a process number for processing the alarm information per minute by a station for displaying the alarm information. The utilization threshold is 1.

ρ represents an average workload of each alarm service station, that is, the alarm system utilization rate. In general, when ρ<1, the alarm system can operate stably. When ρ≥ 1, this indicates that an arrival rate of the alarm information exceeds a service capability of the alarm service stations, and a queue length of the alarm information will grow without bound.

Furthermore, the processor 13 further calculates an idle probability, and the idle probability is calculated according to the following equation:

$${P}_{0} = {[ {\sum }_{n = 0}^{C - 1} \frac{{( λ / μ )}^{n}}{n !} + \frac{{( λ / μ )}^{c}}{c !} \times \frac{1}{1 - ρ} ]}^{- 1}$$

P₀ indicates the idle probability. The idle probability means a probability that there is no alarm in the alarm system, that is, a probability that all alarm service stations have no alarm being displayed.

In addition, the processor 13 further calculates an average number of the alarm information in a queue and an average number of the alarm information in the alarm system. The average number of the alarm information in the queue is calculated according to the following equation:

$${L}_{q} = \frac{{( λ / μ )}^{c} \times ρ}{c ! \times {( 1 - ρ )}^{2}} \times {P}_{0}$$

L_{q} indicates the average number of the alarm information in the queue. The average number of the alarm information in the queue represents an alarm number waiting for displaying in the alarm system, excluding the alarm information that are currently displaying.

The average number of the alarm information in the alarm system is calculated according to the following equation:

$$L = {L}_{q} + \frac{λ}{μ}$$

L indicates the average number of the alarm information in the alarm system. The average number of the alarm information in the alarm system represents an average number including the alarm information in the queue and the alarm information that are currently displaying.

The processor 13 further calculates an average waiting time of the alarm information in the queue and an average stay time of the alarm information in the alarm system. The average waiting time of the alarm information in the queue is calculated according to the following equation:

$${W}_{q} = \frac{{L}_{q}}{λ}$$

W_{q} indicates the average waiting time of the alarm information in the queue. The average waiting time of the alarm information in the queue refers to an average time that an alarm needs to wait after entering the alarm system before its display begins.

Moreover, the average stay time of the alarm information in the alarm system is calculated according to the following equation:

$$W = {W}_{q} + \frac{1}{μ}$$

W indicates the average stay time of the alarm information in the alarm system. The average stay time of the alarm information in the alarm system represents an average time from the time when the alarm information enters the alarm system to the time when the alarm information leaves the alarm system, including both alarm waiting time and alarm display time.

For example, assume that the alarm system receives 12 alarms per minute, that is, λ=12. The display time of each alarm is 10 seconds, such that the alarm system can process 6 alarms per minute, that is, μ=6. The alarm system can simultaneously display 1 alarm, that is, c=1. Therefore, the alarm system utilization rate is:

$$ρ = \frac{λ}{c \times μ} = \frac{12}{1 \times 6} = 2$$

Since the alarm system utilization rate ρ≥1, this means that the service station cannot keep up with the arrival rate, resulting in an unstable system. Namely, this will cause the queue length in the alarm system to approach infinity or the alarm waiting time to approach infinity. In this case, the generated alarm information may include:

Increasing the number of alarm service stations, that is, increasing c to reduce a system load.

Reducing a display time of each alarm, that is, increasing a service efficiency of each alarm service station, for example, increasing u to increase a service rate.

Displaying only high-level alarms and filtering out medium-level and low-level alarms to reduce the number of the arrivals of the alarms, that is, reducing A to reduce the arrival rate.

By adjusting according to the above alarm information until the alarm system utilization rate ρ<1, the alarm system can operate stably.

For example, assume that the alarm system receives 12 alarms per minute, that is, λ=12. Further, the display time of each alarm is reduced to 6 seconds, such that the alarm system can process 10 alarms per minute, that is, μ=10. Moreover, the number of alarm service stations is increased, such as the alarm system can simultaneously display 2 alarms, that is, c=2. Therefore, after the adjustment, the alarm system utilization rate is:

$$ρ = \frac{λ}{c \times μ} = \frac{12}{2 \times 10} = 0.6$$

Namely, ρ=0.6, which means that two service stations of the alarm system are in a busy state for 60% of a total time.

Furthermore, the idle probability P₀ is calculated as follows:

$${P}_{0} = {[ {\sum }_{n = 0}^{C - 1} \frac{{( λ / μ )}^{n}}{n !} + \frac{{( λ / μ )}^{c}}{c !} \times \frac{1}{1 - ρ} ]}^{- 1} = {[ {\sum }_{n = 0}^{1} \frac{{( 12 / 10 )}^{n}}{n !} + \frac{{( 12 / 10 )}^{2}}{2 !} \times \frac{1}{1 - 0.6} ]}^{- 1} = 0.25$$

Namely, P₀=0.25, which means that the alarm system has a 25% probability of being idle.

The average number of the alarm information in the queue L_{q} is calculated as follows:

$${L}_{q} = \frac{{( λ / μ )}^{c} \times ρ}{c ! \times {( 1 - ρ )}^{2}} \times {P}_{0} = \frac{{( 12 / 10 )}^{2} \times 0.6}{2 ! \times {( 1 - 0.6 )}^{2}} \times 0.25 = 0.675$$

Namely, L_{q}=0.675, which means that there are approximately 0.675 alarms waiting in the queue on average.

The average number of the alarm information in the alarm system L is calculated as follows:

$$L = {L}_{q} + \frac{λ}{μ} = 0.675 + \frac{12}{10} = 1.875$$

Namely, L=1.875, which means that there are about 1.875 alarms in total within the alarm system. The average number of the alarm information in the alarm system L includes both queuing and displaying alarms.

The average waiting time of the alarm information in the queue W_{q} is calculated as follows:

$${W}_{q} = \frac{{L}_{q}}{λ} = \frac{0.675}{12} = 0.0563$$

Namely, W_{q}=0.0563, which means that the average waiting time of the alarm information in the queue W_{q} is 0.0563 minutes, or approximately 3.38 seconds.

The average stay time of the alarm information in the alarm system W is calculated as follows:

$$W = {W}_{q} + \frac{1}{μ} = 0.0563 + \frac{1}{10} = 0.1563$$

Namely, W=0.1563, which means that the average stay time of the alarm information in the alarm system W is 0.1563 minutes, or approximately 9.38 seconds.

Summarizing various parameters of the alarm system after the adjustment, the alarm system utilization rate ρ is 60%, the average number of the alarm information in the queue L_{q} is 0.675, the average number of the alarm information in the alarm system L is 1.875, the average waiting time of the alarm information in the queue W_{q} is 0.0563 minutes or approximately 3.38 seconds, and the average stay time of the alarm information in the alarm system Wis 0.1563 minutes or approximately 9.38 seconds.

The above-mentioned various parameters of the alarm system show that the system load is moderate, the alarm waiting time is relatively short, and the service efficiency is improved.

In summary, the physiological information application device 10 is an application platform that allows for installation and uninstallation of applications. Therefore, the physiological information application device 10 can provide a mechanism for exchanging data and for monitoring communication status within the alarm system. Further, since the physiological data are not transferred to an external information device, a risk of data leakage can be reduced, and data confidentiality can be increased. Furthermore, the physiological information application device 10 requires a friendly authentication mechanism to obtain a right to exchange data, further preventing the intrusions of the malicious programs. The physiological information application device 10 also features the star topology architecture and tree topology architecture, further increasing flexibility of the physiological information application device 10. Moreover, an alarm mechanism of the physiological information application device 10 can handle alarms triggered by the application, thereby enhancing functionality of the physiological information application device 10.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A physiological information application platform, comprising a physiological information application device; wherein the physiological information application device comprises:
a sensing signal receiver, communicatively connected to a plurality of sensors for receiving a plurality of physiological data;
a memory, storing at least one server application program and at least one client application program; and
a processor, connected to the sensing signal receiver and the memory for accessing the at least one server application program and the at least one client application program;
wherein the processor executes the at least one server application program to collect the physiological data;
wherein the processor further executes the client application program to access the physiological data collected by executing the at least one server application program, and to generate application program information according to the physiological data.

2. The physiological information application platform as claimed in claim 1, wherein the physiological information application device further comprises:
a plurality of built-in sensors; wherein the sensing signal receiver is connected to the built-in sensors, and receives the physiological data generated by the built-in sensors.

3. The physiological information application platform as claimed in claim 1, wherein the sensing signal receiver is communicatively connected to a plurality of external sensors, and receives the physiological data generated by the external sensors.

4. The physiological information application platform as claimed in claim 1, wherein the physiological information application device further comprises:
a system communicator, communicatively connected to a hospital information system; wherein the processor is further connected to the system communicator;
wherein the at least one client application program at least comprises a first client application program;
wherein the processor executes the first client application program to access the physiological data collected by executing the server application program, and the processor generates first application program information based on the physiological data;
wherein the processor further transmits the first application program information to the hospital information system through the system communicator.

5. The physiological information application platform as claimed in claim 1, wherein the physiological information application device further comprises:
a file receiver, communicatively connected to an authorization device for receiving an authorization encrypted file;
wherein the memory further stores a friendly application program;
wherein the processor is connected to the file receiver to receive the authorization encrypted file, and the processor further decrypts the authorization encrypted file to generate authorization verification information;
wherein the processor accesses the friendly application program, the processor generates friendly application program information according to the friendly application program, and the processor further determines whether the authorization verification information is same as the friendly application program information;
wherein when the authorization verification information is same as the friendly application program information, the processor executes the friendly application program to access the physiological data collected by executing the at least one server application program, and generates the application program information according to the physiological data.

6. The physiological information application platform as claimed in claim 1, wherein the at least one client application program stored in the memory of the physiological information application device comprises multiple client application programs, and the multiple client application programs at least comprise:
a first client application program;
a second client application program;
wherein the processor executes the first client application program to access the physiological data collected by executing the at least one server application program, and the processor further generates first application program information according to the physiological data;
wherein the processor executes the second client application program to access the physiological data collected by executing the at least one server application program, and the processor generates second application program information according to the physiological data.

7. The physiological information application platform as claimed in claim 1, wherein the at least one client application program stored in the memory of the physiological information application device comprises multiple client application programs, and the multiple client application programs at least comprise:
a first client application program;
a second client application program;
wherein the processor executes the first client application program to access the physiological data collected by executing the at least one server application program, and the processor generates first application program information, according to the physiological data;
wherein the processor executes the second client application program to access the first application program information generated by executing the first client application program, and the processor generates second application program information according to the first application program information.

8. The physiological information application platform as claimed in claim 7, wherein the multiple client application programs stored in the memory of the physiological information application device further comprise:
a third client application program;
a fourth client application program;
wherein the processor executes the third client application program to access the first application program information generated by executing the first client application program, and the processor generates third application program information according to the first application program information;
wherein the processor executes the fourth client application program to access the third application program information generated by executing the third client application program, and the processor generates the third application program information according to the third application program information.

9. The physiological information application platform as claimed in claim 1, wherein the physiological information application device further comprises:
an alarm display, connected to the processor;
wherein the memory further stores alarm system parameter information;
wherein the application program information generated by the processor is alarm information;
wherein the processor calculates an alarm system utilization rate according to a number of occurrences of the alarm information generated within each unit time and the alarm system parameter information;
wherein when the alarm system utilization rate is greater than a utilization threshold, the processor generates the alarm information according to the alarm system utilization rate, and the processor transmits the alarm information to the alarm display.

10. The physiological information application platform as claimed in claim 9, wherein the alarm system utilization rate is calculated according to the following equation:
$$ρ = \frac{λ}{c \times μ} ;$$
wherein ρ is the alarm system utilization rate, λ is the number of the occurrences of the alarm information generated per minute, c is a display number of simultaneously displaying the alarm information by the alarm display, and μ is a process number for processing the alarm information per minute by a station for displaying the alarm information;
wherein the utilization threshold is 1;
wherein the processor further calculates an idle probability according to the following equation:
$${P}_{0} = {[ {\sum }_{n = 0}^{C - 1} \frac{{( λ / μ )}^{n}}{n !} + \frac{{( λ / μ )}^{c}}{c !} \times \frac{1}{1 - ρ} ]}^{- 1} ;$$
wherein P₀ is the idle probability;
wherein the processor further calculates an average number of the alarm information in a queue and an average number of the alarm information in the alarm system, and the average number of the alarm information in the queue is calculated according to the following equation:
$${L}_{q} = \frac{{( λ / μ )}^{c} \times ρ}{c ! \times {( 1 - ρ )}^{2}} \times {P}_{0} ;$$
wherein L_{q} is the average number of the alarm information in the queue;
wherein the average number of the alarm information in the alarm system is calculated according to the following equation:
$$L = {L}_{q} + \frac{λ}{μ} ;$$
wherein L is the average number of the alarm information in the alarm system;
wherein the processor further calculates an average waiting time of the alarm information in the queue and an average stay time of the alarm information in the alarm system, and the average waiting time of the alarm information in the queue is calculated according to the following equation:
$${W}_{q} = \frac{{L}_{q}}{λ} ;$$
wherein W_{q} is the average waiting time of the alarm information in the queue;
wherein the average stay time of the alarm information in the alarm system is calculated according to the following equation:
$$W = {W}_{q} + \frac{1}{μ} ;$$
wherein W is the average stay time of the alarm information in the alarm system.
